(19) United States
(12) Patent Application Publication
(10) Pub. No.: US 2023/0173288 A1
(43) Pub. Date: Jun. 8, 2023

(54) COMPACT DC SYSTEM FOR DELIVERING A SQUARE WAVE AC SIGNAL

(71) Applicant: Novocure GmbH, Root D4 (CH)

(72) Inventor: Richard Deslauriers, Woodbury, CT (US)

(21) Appl. No.: 18/062,161

(22) Filed: Dec. 6, 2022

Related U.S. Application Data
(60) Provisional application No. 63/286,687, filed on Dec. 7, 2021.
(60) Provisional application No. 63/313,850, filed on Feb. 25, 2022.

(51) Int. Cl. A61N 1/40; A61N 1/36; A61N 1/04

(52) U.S. Cl. CPC A61N 1/40; A61N 1/36002; A61N 1/0472

(57) ABSTRACT

Apparatus and methods for imposing electric fields through a target region in a body of a patient are described. Generally, the apparatus may include an electric field generator having a first circuit generating a first output signal having a positive voltage; a second circuit generating a second output signal having a negative voltage, and a processor executing processor executable instructions to alternatingly enable the first output signal, and the second output signal to a first port and a second port to generate an alternating current square wave in a frequency range from 50 kHz to 1 MHz.

### CROSS-REFERENCE TO RELATED APPLICATIONS

This application is a non-provisional application claiming benefit to the U.S. Provisional Application No. 63/286,687 filed on Dec. 7, 2021, and U.S. Provisional Application No. 63/313,850 filed on Feb. 25, 2022. The entire content of each of the above-referenced applications are hereby incorporated herein by reference in their entirety.

### BACKGROUND

TTFields therapy is a proven approach for treating tumors. For example, using the Optune® system for delivering tumor treating fields (i.e., TTFields), the TTFields are delivered to patients via four transducer arrays placed on the patient's skin in close proximity to a tumor. The transducer arrays are arranged in two pairs, and each transducer array is connected via a multi-wire cable to an electric field generator. The electric field generator (a) sends an AC current through one pair of arrays during a first period of time; then (b) sends an AC current through the other pair of arrays during a second period of time; then repeats steps (a) and (b) for the duration of the treatment.

### SUMMARY OF THE DISCLOSURE

It is desirable for the electric field generator to be portable and as small as possible to provide greater comfort and accessibility to the tumor treating fields and thereby avoid interference with the patient's lifestyle. A need exists for a portable electric field generator for imposing electric fields through a target region in a body of a patient that is smaller, lower cost, and more efficient than conventional electric field generators. In some embodiments, the portable electric field generator includes a portable housing sized to fit within a pants pocket, e.g., within a range of 5-10 cm in width, 12-17 cm in length, and ½-3 cm in thickness. This allows the portable electric field generator to be carried by a patient in their pocket or purse, for example, and also provides AC current through the transducer arrays for an extended period of time (e.g., 4-5 hours with a 30V 90 watt hour battery) preferably solely with battery power.

More particularly, disclosed herein is a system for generating TTFields, comprising a first port operable to receive a first lead of a first transducer array, a second port operable to receive a second lead of a second transducer array; and an electric field generator having a first circuit generating a first output signal having a positive voltage; a second circuit generating a second output signal having a negative voltage, and a processor executing processor executable instructions to alternatingly enable the first output signal, and the second output signal to the first port and the second port to generate an alternating current square wave in a frequency range from 50 kHz to 1 MHz. Preferably the first output signal is a first direct current square wave having a duty cycle between 15% to 40% and varying between ground and the positive voltage, and the second output signal is a second direct current square having a duty cycle between 15% to 40% and varying between ground and the negative voltage. The processor synchronizes the first output signal and the second output signal so as to generate the alternating current square wave in the form of a modified square wave, as discussed in more detail below. The first output signal and the second output signal may be approximately 180 degrees out of phase.

In some embodiments, the system includes a battery. In some of these embodiments, the first output signal has a positive amplitude (e.g., the positive voltage) within 5% of the battery voltage, and is ideally equal to the battery voltage. In these embodiments, the second output signal has a negative amplitude (e.g., the negative voltage) within 5% of a negative of the battery voltage, and is ideally equal to a negative of the battery voltage. For example, if the battery voltage is 30V, the positive amplitude may be in a range of +28.5 V to +31.5 V and the negative amplitude may be in a range of −28.5 V to −31.5V. In some embodiments, the processor, the first circuit, the second circuit and the battery are contained within the portable housing. The first port and the second port can be located on an exterior of the portable housing so that leads from the transducer array can plug into the first port and the second port.

When a first transducer array and a second transducer array are applied to skin of the patient, and the electric signal is supplied to the first transducer array and the second transducer array, a tumor treating electric field is applied to the patient and current flows between the first transducer array and the second transducer array. In this instance, impedance between the first transducer array and the second transducer array is due to the electrical connection of the first transducer array and the second transducer array to the patient, and also due to the patient's body.

Conventionally, the electric field generator for creating TTFields in a patient sends an electric signal at a maximum power and the first and second conventional transducer arrays are intended to be continuously worn by the patient for 2-4 days before removal for hygienic care and re-shaving (if necessary), followed by reapplication with a new set of conventional transducer arrays. In this time period, the patient's hair can grow and push the conventional electrode arrays away from the patient's skin and the patient's skin may produce oils thereby increasing impedance in the electrical connection between the conventional transducer arrays and the patient's skin. This increase in impedance can increase the temperature of the conventional transducer arrays. The impedance can be within a range of 30 to 160 Ohms. When the temperature of the conventional transducer array reaches a predetermined temperature of 41 degrees Celsius, the conventional electric field generator, in communication with one or more temperature sensor in the conventional transducer arrays may reduce the current and/or reduce the voltage applied to the conventional transducer arrays which in turn causes a reduction in the tumor treating fields applied to the patient. This requires complex processing to constantly monitor the temperature of the conventional transducer arrays as well as additional wiring to communicate temperature signals from each temperature sensor to the conventional electric field generator.

In some embodiments, the battery voltage and the amplitudes of the first output signal and the second output signal are at a level that will avoid heating the transducer arrays above a comfortability threshold in a range from 36-42 degrees centigrade when the impedance through the patient's body is in the range of 20 to 160 Ohms. In some embodiments the battery voltage and the amplitudes of the first output signal and the second output signal can be in a range from 20-40 Volts, and is more preferably 30 Volts. When the amplitudes of the first output signal and the second output signal are 30 Volts, then the current that flows through the patient can be in a range of 1.5 A-0.1875 A resulting in power within a range of 45 W-5.625 W. Because the battery voltage and the amplitudes of the first output signal and the second output signal are maintained at a level to avoid heating the transducer arrays to an uncomfortable extent (e.g., above 41 degrees centigrade), the electric field generator of the present disclosure can be devoid of any circuit that receives feedback from the transducer arrays, such as temperature readings from temperature sensors, as well as any circuitry to control the voltage and/or current of the first output signal and the second output signal based upon feedback from the transducer arrays. This results in the system being very small, lightweight, and efficient thereby increasing the amount of TTFields that can be delivered to the patient with energy from the battery.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is an exemplary embodiment of a schematic diagram of electrodes as applied to living tissue.

FIG. 2 is an exemplary embodiment of an electronic device configured to generate a TTField constructed in accordance with the present disclosure.

FIG. 3 is a block diagram of an exemplary embodiment of a transducer array constructed in accordance with the present disclosure.

FIG. 4 is a block diagram of another exemplary embodiment of a transducer array constructed in accordance with the present disclosure.

FIG. 5A is a graph showing three different types of waveforms (i.e., a sine-wave, a square wave and a modified square wave) capable of being applied to the transducer arrays and generating TTFields within a patient.

FIG. 5B is a graph showing two different types of waveforms (i.e., a sine-wave, and a modified sine wave) capable of being applied to the transducer arrays and generating TTFields within a patient.

FIG. 6A is a block diagram of an exemplary embodiment of an electric field generator constructed in accordance with the present disclosure.

FIG. 6B is a block diagram of another exemplary embodiment of an electric field generator constructed in accordance with the present disclosure.

FIG. 7 is a graph showing three waveforms generated by the electric field generator in accordance with the present disclosure.

### DETAILED DESCRIPTION

The TTFields are generally delivered to patients via four transducer arrays placed on the patient's skin conventionally as two orthogonal pairs in locations chosen to best target the tumor. Each transducer array is configured as a set of coupled electrode elements (for example, about 2 cm in diameter) that are interconnected via flex wires. Commonly, each electrode element includes a ceramic disk that is sandwiched between a skin interface layer that may include an electrically conductive medical gel and an adhesive tape. When placing the arrays on the patient, the medical gel adheres to the contours of the patient's skin and ensures good electrical contact of the device with the body. The adhesive tape holds the entire array in place on the patient as the patient goes about their daily activities.

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular.

All of the assemblies, systems, kits, and/or methods disclosed herein can be made and executed without undue experimentation in light of the present disclosure. While the assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, or greater numbers of compounds. The term “plurality” refers to “two or more.”

The use of the term “at least one” will be understood to include one as well as any quantity more than one. The use of ordinal number terminology (i.e., “first,” “second,” “third,” “fourth,” etc.) is solely for the purpose of differentiating between two or more items and does not imply any sequence or order or importance to one item over another or any order of addition.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” in various places in the specification is not necessarily all referring to the same embodiment, for example.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “patient” as used herein encompasses any mammals including human and veterinary subjects. “Mammal” for purposes of treatment refers to any animal classified as a mammal, including (but not limited to) humans, domestic and farm animals, nonhuman primates, and any other animal that has mammary tissue. In some embodiments, the term “patient” may apply to a simulation mannequin for use in teaching, for example.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

The term “TTField”, as used herein, means tumor treating field, e.g., low intensity (e.g., 1-4 V/cm) alternating electric fields of medium frequencies (about 50 kHz-1 MHz, and more preferably from about 100 kHz-300 kHz) that when applied to a conductive medium, such as a human body, via electrodes may be used, for example, to treat tumors as described in U.S. Pat. Nos. 7,016,725, 7,089,054, 7,333,852, 7,565,205, 7,805,201, and 8,244,345 by Palti (each of which is incorporated herein by reference) and in a publication by Kirson (see Eilon D. Kirson, et al., Disruption of Cancer Cell Replication by Alternating Electric Fields, Cancer Res. 2004 64:3288-3295).

As used herein, the term TTSignal is an electrical signal that, when received by electrodes applied to a conductive medium, such as a human body, causes the electrodes to generate the TTField described above. The TTSignal is often an AC electrical signal.

Referring now to the drawings and in particular to FIG. 1, shown therein is a diagram of an exemplary embodiment of a dividing cell 10, under the influence of external TTFields (e.g., alternating fields in the frequency range of about 50 kHz to about 1 MHz), generally indicated as lines 14, generated by a first electrode 18a having a negative charge and a second electrode 18b having a positive charge. Further shown are microtubules 22 that are known to have a very strong dipole moment. This strong polarization makes the microtubules 22, as well as other polar macromolecules and especially those that have a specific orientation within the cell 10 or its surroundings, susceptible to electric fields. The positive charges of the microtubules 22 are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions. In one embodiment, the cells go through electroporation, that is, DNA or chromosomes are introduced into the cells using a pulse of electricity to briefly open pores in the cell membranes.

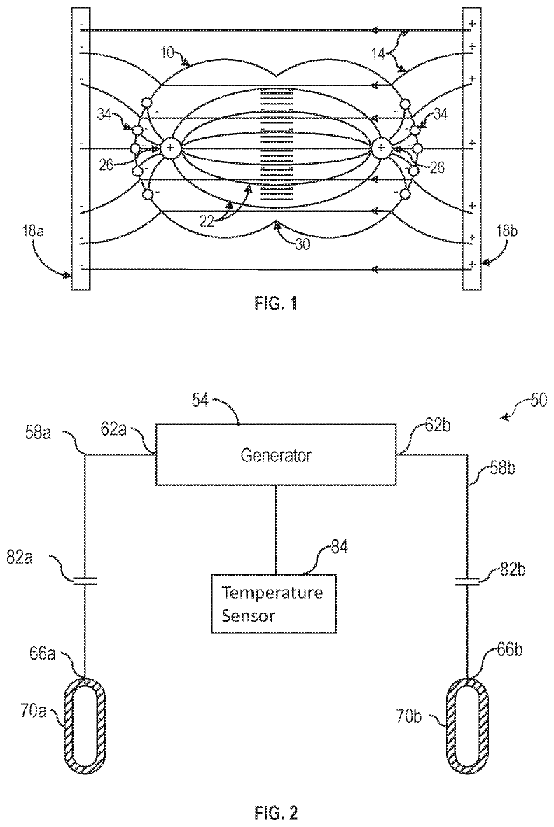

Turning now to FIG. 2, the TTFields described above that have been found to advantageously destroy tumor cells may be generated by an electronic apparatus 50. FIG. 2 is a simple schematic diagram of the electronic apparatus 50 illustrating major components thereof. The electronic apparatus 50 includes an electric field generator 54 and a pair of conductive leads 58, including first conductive lead 58a and second conductive lead 58b. The first conductive lead 58a includes a first end 62a and a second end 66a. The second conductive lead 58b includes a first end 62b and a second end 66b. The first end 62a of the first conductive lead 58a is conductively attached to the electric field generator 54 and the first end 62b of the second conductive lead 58b is conductively attached to the electric field generator 54. The electric field generator 54 generates desirable electric signals (TT signals) in the shape of waveforms or trains of pulses as an output. The second end 66a of the first conductive lead 58a is connected to a transducer array 70a and the second end 66b of the second conductive lead 58b is connected to a transducer array 70b. Both of the transducer array 70a and the transducer array 70b receive the electric signals (e.g., TT signals, wave forms). The transducer array 70a and the transducer array 70b, receiving the electric signals, causes an electrical current to flow between the transducer array 70a and the transducer array 70b. The electrical current generates an electric field (i.e., TTField), having a frequency and an amplitude, to be generated between the transducer array 70a and the transducer array 70b.

While the electronic apparatus 50 shown in FIG. 2 comprises only two transducer arrays 70 (the transducer array 70a and the transducer array 70b), in some embodiments, the electronic apparatus 50 may comprise more than two transducer arrays 70.

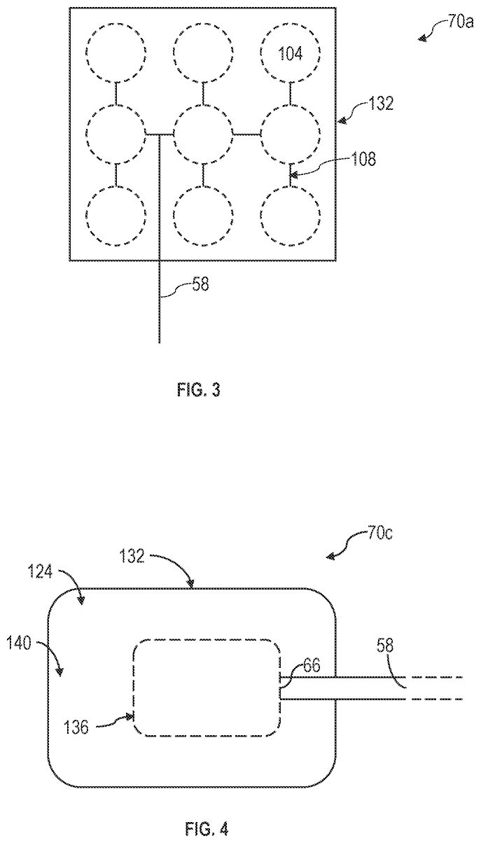

The electric field generator 54 generates an alternating voltage wave form at frequencies in the range from about 50 kHz to about 1 MHz (preferably from about 100 kHz to about 300 kHz) (i.e., the TTFields). The voltages may be such that an electric field intensity in tissue within the treatment area is in the range of about 0.1 V/cm to about 10 V/cm. To achieve this field, the potential difference between the two conductors 18 (e.g., the electrode element 104 shown in FIG. 3 and described in detail below) in each of the transducer array 70a or the transducer array 70b is determined by the relative impedances of the system components, e.g., a fraction of the electric field on each component is given by that component's impedance divided by a total circuit impedance.

In certain particular (but non-limiting) embodiments, the transducer array 70a and the transducer array 70b generate an alternating electric current and field within a target region of a patient. The target region typically comprises at least one tumor, and the generation of the alternating electric current and field selectively destroys or inhibits growth of the tumor. The alternating electric current and field may be generated at any frequency that selectively destroys or inhibits growth of the tumor, e.g., TTField.

The pair of transducer arrays 70a and 70b, as described herein, are externally applied to a patient, that is, are generally applied to the patient's skin, in order to apply the electric current, and electric field (TTField) thereby generating current within the patient's tissue. Generally, the pair of transducer arrays 70a and 70b are placed on the patient's skin by a user such that the electric field is generated across patient tissue within a treatment area. TTFields that are applied externally can be of a local type or widely distributed type, for example, the treatment of skin tumors and treatment of lesions close to the skin surface.

In one embodiment, the user may be a medical professional, such as a doctor, nurse, therapist, or other person acting under the instruction of a doctor, nurse, or therapist. In another embodiment, the user may be the patient, that is, the patient may place the transducer array 70a and the transducer array 70b on their treatment area.

As discussed above, in some embodiments, the electric field generator 54 is configured to avoid applying sufficient power to the transducer arrays 70a and 70b to cause a temperature of the transducer arrays 70a and 70b to exceed a comfortability threshold. In one embodiment, the comfortability threshold is the temperature at which a patient would be made uncomfortable while using the transducer array 70a and the transducer array 70b. In one embodiment, the comfortability threshold is a temperature within a range from 36-42 degrees Celsius. In one embodiment, the comfortability threshold is a temperature of between about 39 degrees Celsius and 42 degrees Celsius, or a specific selected temperature between about 39 degrees Celsius and 42 degrees Celsius, such as, for example, 41 degrees Celsius. In other embodiments, the electronic apparatus 50 is provided with a temperature sensor 84 providing temperature signals to the electric field generator 54 so that the electric field generator 54 can determine whether or not the transducer arrays 70a and/or 70b are above or below the comfortability threshold and vary the power being supplied to the transducer arrays 70a and 70b as discussed below.

The conductive leads 58 may be standard isolated conductors with a flexible metal shield, preferably grounded thereby preventing spread of any electric field generated by the conductive leads 58. The transducer array 70a and the transducer array 70b may have specific shapes and positioning so as to generate the TTField of a desired configuration, direction, and intensity at the treatment area and only at that treatment area so as to focus the treatment.

The specifications of the electronic apparatus 50 as a whole and its individual components are largely influenced by the fact that at the frequency of the TTFields (50 kHz-1 MHz), living systems behave according to their “Ohmic”, rather than their dielectric properties.

In one embodiment, to protect the patient from any current due to DC voltage or DC offset voltage passing through the patient, leads 58a and 58b may include a DC blocking component, such as blocking capacitor 82a and blocking capacitor 82b, to block DC current from passing to the transducer array 70a and the transducer array 70b. The blocking capacitors 82a and 82b pass AC voltage to the transducer array 70a and the transducer array 70b, and also prevent any DC voltage or DC offset generated by the electric field generator 54 or otherwise present in the electrical signal from passing to or through the patient. The blocking capacitors 82a and 82b can prevent electrolysis due to DC offsets or DC voltage. In one embodiment, the blocking capacitors 82a and 82b are non-polarized capacitors. In one embodiment, the blocking capacitors 82a and 82b have a capacitance of about 1 μF. In one embodiment, the blocking capacitor is a “Goldmax, 300 Series, Conformally Coated, X7R Dielectric, 25-250 VDC (Commercial Grade)” leaded non-polarized ceramic capacitor by KEMET Electronics Corporation (Fort Lauderdale, Fla.).

In other embodiments, the blocking capacitor 82a and the blocking capacitor 82b may be components of the electric field generator 54, that is, the blocking capacitor 82a and the blocking capacitor 82b may be integrated into the electric field generator 54 such that prior to the electrical signal being passed into the leads 58a and 58b, the electrical signal passes through the blocking capacitors 82a and 82b, respectively.

Referring now to FIG. 3, shown therein is a diagram of an exemplary embodiment of the transducer array 70a constructed in accordance with the present disclosure. The transducer array 70a includes one or more electrode element 104. As shown in FIG. 3, the transducer array 70a is configured as a set of one or more electrode elements 104. The transducer array 70a may utilize electrode elements 104 that are capacitively coupled. In the example shown in FIG. 3, the transducer array 70a is configured as multiple electrode elements 104 (for example, about 2 cm in diameter) that are interconnected via flex wires 108. Each electrode element 104 may include a ceramic disk positioned between an electrode layer and a skin-facing surface of the transducer array 70a. In one embodiment, the transducer array 70a includes an outer peripheral edge 132.

Alternative constructions for the transducer array 70a may be used, including, for example ceramic elements that are disc-shaped, ceramic elements that are not disc-shaped, and non-ceramic dielectric materials positioned between the electrode layer and a skin-facing surface of the transducer array 70a. Examples of non-ceramic dielectric materials positioned over a plurality of flat conductors include: polymer films disposed over transducer arrays on a printed circuit board or over flat pieces of metal. The transducer array 70a may utilize electrode elements 104 that are not capacitively coupled. In this situation, each electrode element 104 of the transducer array 70a would be implemented using a region of a conductive material that is configured for placement against a patient's body, with no insulating dielectric layer disposed between the electrode elements 104 and the body. Examples of the conductive material include a conductive film, a conductive fabric, and a conductive foam. Other alternative constructions for implementing the transducer array 70a may also be used, as long as they are capable of delivering TTFields to the patient's body. Optionally, a gel layer may be disposed between the transducer array 70a and the patient's body in any of the embodiments described herein. The transducer array 70b can be constructed in a similar manner as the transducer array 70a.

Referring now to FIG. 4, shown therein is a top plan view of an exemplary embodiment of a transducer array 70c. The transducer array 70c is an exemplary embodiment of the transducer array 70a or the transducer array 70b. The transducer array 70c may be provided with a top 124, a bottom, an outer peripheral edge 132, and an electrode element 136 bounded by the outer peripheral edge 132. As shown, the transducer array 70c is connected to the second end 66 of the conductive lead 58. The transducer array 70c is constructed so as to have sufficient flexibility and to be able to conform to a portion of the patient, such as a portion of the patient's head, the patient's knee, the patient's elbow, or the like. The transducer array 70c may also be constructed such that the electrode element 136 is continuous, and extends to the outer peripheral edge 132. In the example shown, the transducer array 70c is provided with a rectangular shape, or substantially rectangular shape having rounded vertices. However, it should be understood that the transducer array 70c can be provided with any type of shape such as a polygon, circle, or fanciful shape. Further, the transducer array 70c may be constructed such as to be cut and/or shaped at a point of use so as to be custom fitted for a particular part of a particular patient.

In one embodiment, the transducer array 70c is provided with a durable topcoat layer 140 as the top 124. The durable topcoat layer 140 may be a non-woven, non-conductive fabric. The durable topcoat layer 140 provides a safe handling surface for the transducer array 70c to electrically isolate the electrode element 136 from the top 124 of the transducer array 70c. In some embodiments, the durable topcoat layer 140 is colored to match or approximate the skin color of the patient.

Hereinafter, the transducer array 70a, transducer array 70b, and transducer array 70c may be referred to singly as transducer array 70 or plurally as transducer arrays 70. Unless otherwise specified, reference to the transducer array 70 should be understood to refer to any one of the transducer array 70a, transducer array 70b, and transducer array 70c and reference to the transducer arrays 70 should be understood to refer to two or more of the transducer array 70a, the transducer array 70b, and/or the transducer array 70c, or any combination thereof.

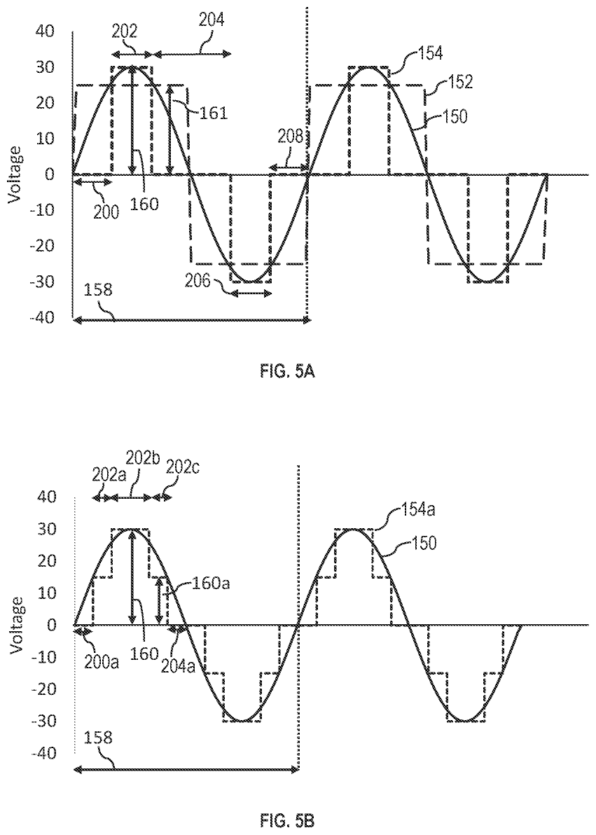

FIG. 5A is a graph showing three different types of waveforms (i.e., a sine-wave 150, a square wave 152 and a modified square-wave 154) capable of being applied to the transducer arrays 70, and generating TTFields within a patient. The sine-wave 150, the square wave 152 and the modified square-wave 154 have a period 158, which may be 20 milliseconds, for example. The sine-wave 150 and the modified square-wave 154 have an amplitude 160, which as discussed above may be within 5% of the battery voltage, and is ideally equal to the battery voltage. The square wave 152 having a 50% duty cycle has an amplitude 161 that is less than the amplitude 160 of the sine-wave 150 and the modified square-wave 154. For a same load, the square wave 152 having the 50% duty cycle will deliver twice the power as the sine-wave 150 having the same amplitude. In this case, the square wave 152 has the amplitude 161 that is less than the amplitude 160 so as to deliver the same amount of power as the sine-wave 150. The modified square wave 154 has the amplitude 160, and a duty cycle that is configured to deliver a similar amount of power as the sine-wave 150, but without the circuitry required to generate the sine-wave 150. By generating the square wave 152, or the modified square-wave 154, the electric field generator 54 can deliver more power to the patient with a given battery, than if the electric field generator 54 supplied the sine-wave 150 to the patient because the electric field generator 54 can generate the square wave 152 or the modified square-wave 154 with less circuitry resulting in fewer energy losses. In some embodiments, the amplitude 161 may be in a range of 25-35% less than the amplitude 160.

The square wave 152 is a non-sinusoidal periodic waveform in which the amplitude alternates at a steady frequency between fixed minimum (e.g., −30V) and maximum values (e.g., +30V). In some embodiments, the square wave 152 has a same duration at minimum and maximum. The square wave may have a duty cycle of 50%, which is the ratio of time that the square wave is in a positive state (e.g., at the maximum value) relative to the period 158. In an ideal square wave, the transitions between minimum and maximum are instantaneous. At +30V and −30V maximum and minimum values, and 200 kHz frequency, the square wave 152, however, when used to generate TTFields, does not provide an unwanted electrical sensation to the patient when the TTFields are applied. The modified square-wave 154 is a non-sinusoidal waveform that in some embodiments includes three sequential components, i.e., a positive square wave, a ground voltage level, and a negative square wave. The modified square wave is similarly periodic but includes asymmetric waves, i.e., duty cycles other than 50%. In the example shown, the modified square wave 154 has a duty cycle in a range of 18-22% so as to deliver power in a similar manner as the sine-wave 150 but over a longer duration for a given battery. In some embodiments, the duty cycle of the modified square-wave 154 can vary between 15% to 40%.

FIG. 5B is a graph showing two different types of waveforms (i.e., a sine-wave 150 and a modified square wave 154a) capable of being applied to the transducer arrays 70, and generating TTFields within a patient. The sine-wave 150 and the modified square wave 154a are similar to the sine-wave 150 and the modified square-wave 154 of FIG. 5A with the exception of the modified square wave 154a includes an intermediate amplitude 160a held for a predetermined period. The sine-wave 150 and the modified square wave 154a have a period 158, which may be 20 milliseconds, for example. The sine-wave 150 and the modified square-wave 154a have an amplitude 160, which as discussed above may be within 5% of the battery voltage, and is ideally equal to the battery voltage. The modified square-wave 154a further includes the intermediate amplitude 160a which may be within 30%-70% of the battery voltage, and is ideally equal to a voltage of a first battery component as discussed below.

The modified square wave 154a has the intermediate amplitude 160a, the amplitude 160, and a duty cycle that is configured to deliver a similar amount of power as the sine-wave 150, but without the circuitry required to generate the sine-wave 150. By generating the modified square-wave 154a, an electric field generator 54′ can deliver more power to the patient with a given battery, than if the electric field generator 54′ supplied the sine-wave 150 to the patient because the electric field generator 54′ can generate the modified square-wave 154a with less circuitry resulting in fewer energy losses. In some embodiments, the intermediate amplitude 160a may be in a range of 25-35% less than the amplitude 160.

The modified square wave 154a is a non-sinusoidal waveform that in some embodiments includes seven sequential components, i.e., a first positive square wave having the intermediate amplitude 160a, a second positive square wave having the amplitude 160, a third positive square wave having the intermediate amplitude 160a, a ground voltage level, a first negative square wave having a negative of the intermediate amplitude 160a, a second negative square wave having a negative of the amplitude 160, and a third negative square wave having a negative of the intermediate amplitude 160a. The modified square wave 154a is similarly periodic but includes asymmetric waves, i.e., duty cycles other than 50%. In the example shown, the modified square wave 154a may have a different duty cycle than the modified square-wave 154 (FIG. 5A) so as to deliver power in a similar manner as the sine-wave 150. In some embodiments, the duty cycle of the modified square wave 154a can vary between 15% to 40%.

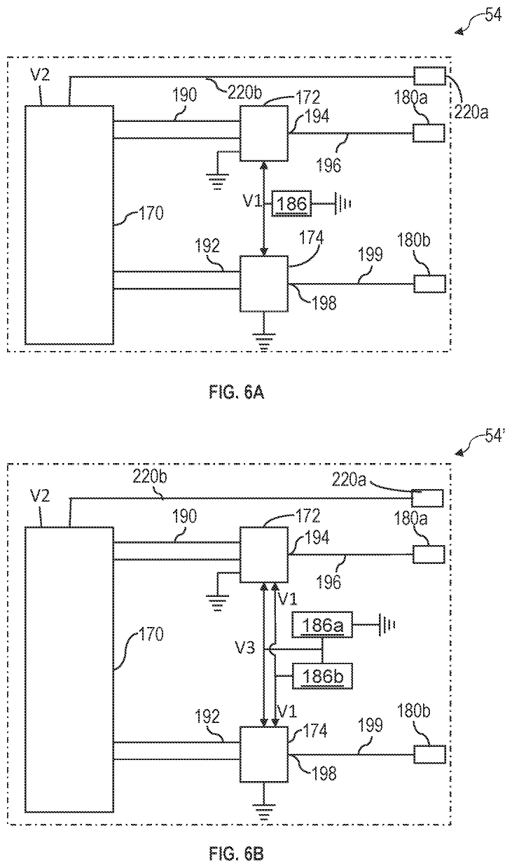

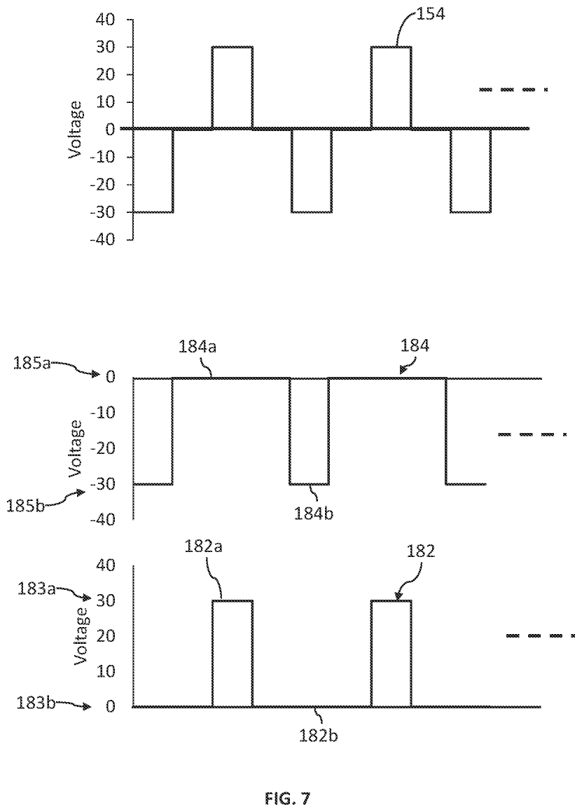

FIG. 6A is a block diagram of an exemplary embodiment of the electric field generator 54 constructed in accordance with the present disclosure. The electric field generator 54 includes a processor 170, a first circuit 172, a second circuit 174, a first port 180a, and a second port 180b. The first port 180a is operable to receive the lead 58a of the transducer array 70a. The second port 180b is operable to receive the lead 58b of the transducer array 70b. The first circuit 172 generates a first output signal 182 (See FIG. 7) having a positive voltage 183a. The second circuit 174 generates a second output signal 184 (see FIG. 7) having a negative voltage 185b. The processor 170 executes processor executable instructions to alternatingly enable the first output signal 182, and the second output signal 184 to the first port 180a and the second port 180b, respectively, to generate an alternating current square wave in a frequency range from 50 kHz to 1 MHz. The processor executable instructions can be stored on a non-transitory computer readable medium coupled to the processor 170 via a data bus (internal or external to the processor 170), or a network. Exemplary non-transitory computer readable mediums include random access memory, flash memory, read only memory and the like.

Preferably the first output signal 182 (see FIG. 7) is a first direct current square wave having a duty cycle between 15% to 40% and varying between ground 183b and the positive voltage 183a, and the second output signal 184 (see FIG. 7) is a second direct current square having a duty cycle between 85% to 60% (in a high state) and varying between ground 185a and the negative voltage 185b. The processor 170 synchronizes the first output signal 182 and the second output signal 184 so as to generate the alternating current square wave in the form of the modified square-wave 154. The first output signal 182 may be a first direct current waveform having a first portion 182a having a first voltage and a second portion 182b having a second voltage lower than the first voltage. The second output signal 184 may be a second direct current waveform having a third portion 184a having a third voltage, and a fourth portion 184b having a fourth voltage higher than the third voltage. The first direct current waveform and the second direct current waveform are out of phase such that the first and fourth portions overlap, and the second and third portions overlap.

In some embodiments, the system includes a battery having a voltage V1. The electric field generator 54 may include a DC voltage converter to convert the voltage V1 to a lower voltage V2. The voltage V2 can be supplied to the processor 170. For example, in some embodiments the voltage V2 can be 3.3V.

In some of these embodiments generating the modified square-wave 154, the first output signal 182 has a positive amplitude (e.g., the positive voltage 183a) within 5% of the battery voltage V1, and is ideally equal to the battery voltage V1. In these embodiments, the second output signal 184 has a negative amplitude (e.g., the negative voltage 185b) within 5% of a negative of the battery voltage V1, and is ideally equal to a negative of the battery voltage. For example, if the battery voltage V1 is 30V, the positive amplitude may be in a range of +28.5 V to +31.5 V and the negative amplitude may be in a range of −28.5 V to −31.5 V.

In some embodiments in which the electronic apparatus 50 includes the temperature sensor 84, the electric field generator 54 includes a third port 200a configured to receive a connector electrically connected to the temperature sensor 84. The processor 170 can be electrically connected to the third port 200a via a conductive trace or wire 200b. In these embodiments, the processor 170 receives a series of signals indicative of temperature readings from the temperature sensor 84. In these embodiments, the processor executable instructions have a temperature compensation subroutine to cause the processor 170 to alternatingly enable the first output signal, and the second output signal to the first port 180a and the second port 180b to generate the alternating current square wave in a frequency range from 50 kHz to 1 MHz having at least one non-voltage parameter based upon the temperature reading. As discussed above, the alternating current square wave has a duty cycle. In some embodiments, the non-voltage parameter is the duty cycle, wherein the temperature compensation subroutine causes the processor 170 to vary the duty cycle of the alternating current square wave based upon the at least one temperature reading. The temperature compensation subroutine may cause the processor 170 to reduce the duty cycle when the at least one temperature reading exceeds a predetermined temperature thereby reducing the power being applied to the first transducer array 70a and the second transducer array 70b.

In some embodiments, the battery voltage V1 and the amplitudes of the first output signal 182 and the second output signal 184 are at a level that will avoid heating the transducer arrays 70 above the comfortability threshold notwithstanding the impedance within the patient's body (which may vary in a range of 20 to 160 Ohms). In some embodiments the battery voltage V1 and the amplitudes of the first output signal 182 and the second output signal 184 can be in a range from 20-40 Volts, and is more preferably 30 Volts. When the amplitudes of the first output signal 182 and the second output signal 184 are 30 Volts, then the current that flows through the patient can be in a range of 1.5 A-0.1875 A, resulting in power within a range of 45 W-5.625 W. Because the battery voltage V1 and the amplitudes of the first output signal 182 and the second output signal 184 are maintained at a level to avoid heating the transducer arrays 70 to an uncomfortable extent, the electric field generator 54 of the present disclosure can be devoid of any circuit that receives feedback from the transducer arrays 70, such as temperature readings from temperature sensors, as well as any circuitry to compensate for or measure the temperature or any circuitry to control the voltage and/or current of the first output signal 182 and the second output signal 184 based upon feedback from the transducer arrays 70. This results in the electronic apparatus 50 being very small, lightweight, and efficient thereby increasing the amount of TTFields that can be delivered to the patient with energy from the battery 186.

The first circuit 172 and the second circuit 174 can be half-bridge bipolar switches, such as a UC2950 obtainable from Texas Instruments, Inc. The first circuit 172 may be connected to and receive instructions from the processor 170 via control lines 190. The second circuit 174 may be connected to and receive instructions from the processor 170 via control lines 192. An output 194 of the first circuit 172 is connected to the first port 180a via power line 196. An output 198 of the second circuit 174 is connected to the second port 180b via power line 199.

In one embodiment, such as shown in FIG. 6, the first circuit 172 and the second circuit 174 do not receive a reference signal from an oscillator circuitry. In this embodiment, the electric field generator 54 may be devoid of any oscillator circuitry providing a reference signal to the first circuit 172 and the second circuit 174.

Referring again to FIG. 5A, the processor 170 executes a subroutine (i.e., particular set of processor executable instructions) in a repeated manner so as to generate the modified square-wave 154, preferably without using an amplifier to change the voltage and/or current characteristics of the first output signal 182 or the second output signal 184. Specifically, for each period 158, the subroutine enables the first circuit 172 and the second circuit 174 to supply and hold the ground signal (e.g., ground 183b and ground 185a depicted in FIG. 7) on the power lines 196 and 199 for a non-transitory first predetermined period of time 200, followed by enabling the first circuit 172 to supply and hold the positive voltage 183a (see FIG. 7) on the power line 196 and the second circuit 174 to supply and hold the ground 185a on the power line 199 for a non-transitory second predetermined period of time 202. Then, the subroutine enables the first circuit 172 and the second circuit 174 to supply and hold the ground signals (e.g., ground 183b and ground 185a) on the power lines 196 and 199 for a non-transitory third predetermined period of time 204, followed by the subroutine enabling the second circuit 174 to supply and hold the negative voltage 185b on the power line 199 and the first circuit 172 to supply and hold the ground 183b on the power line 196 for a non-transitory fourth predetermined period of time 206, followed by the subroutine enabling the first circuit 172 and the second circuit 174 to supply and hold the ground signal (e.g., ground 183b and ground 185a) on the power lines 196 and 199 for a non-transitory fifth predetermined period of time 208. This subroutine is then repeated to continuously supply the modified square-wave 154 to the first port 180a and the second port 180b, and thus the transducer arrays 70. In some embodiments, the processor executable instructions do not change the duty cycle of the first output signal 182 and the second output signal 184. In these embodiments, the processor 170 does not receive any temperature related feedback from the transducer arrays 70a and 70b, and the processor executable instructions do not include any instructions to modify the signals provided to the first circuit 172 or second circuit 174 based upon temperature related feedback.

In use, the processor 170 alternately enables the first output signal 182 having the positive voltage 183a from the first circuit 172 and the second output signal 184 having a negative voltage 185b from the second circuit 174 so as to provide an alternating current square wave in a frequency range of 50 kHz and 1 MHz at the first port 180a and the second port 180b of the electric field generator 54. The alternating current square wave is supplied to the transducer arrays 70 that are mounted on a portion of a patient's body adjacent to a tumor to generate the TTFields.

In one embodiment, when the modified square-wave 154 has a frequency of 200 kHz, the modified square-wave 154 has a period 158 of 5 μs. In some embodiments, each predetermined period of time 202-206 and the sum of predetermined period of time 200 and 208, is approximately equal, i.e., approximately 25% of the period 158. For example, when the modified square-wave 154 has the period 158 of 5 μs, each of the predetermined period of time 202-206 and the sum of predetermined period of time 200 and 208, is equal to 1.25 μs. In other embodiments, the predetermined periods of time 202-206 and the sum of predetermined periods of time 200 and 208, is between about 15% and about 30% of the period 158.

FIG. 6B is a block diagram of an exemplary embodiment of an electric field generator 54′ constructed in accordance with the present disclosure. The electric field generator 54′ may be constructed in accordance with the electric field generator 54 described above with reference to FIG. 6A with the exception that the electric field generator 54′ includes a first battery component 186a and a second battery component 186b in place of the battery 186. In this embodiment, the first battery component 186a may deliver a voltage V3 to the first circuit 172 and the second circuit 174 corresponding to the intermediate amplitude 160a (FIG. 5B above) and a voltage V1 to the first circuit 172 and the second circuit 174 corresponding to the amplitude 160 (FIG. 5B above).

In one embodiment, the first circuit 172 may generate a first output signal by outputting a signal at V3, then V1, then V3, then 0 v for a first half of the period 158. The second circuit 174 may generate a second output signal by outputting a signal at −V3, then −V1, then −V3, then 0 v for a second half of the period 158. The processor 170 executes processor executable instructions to alternatingly enable the first output signal, and the second output signal to the first port 180a and the second port 180b, respectively, to generate an alternating current square wave in the form of the modified square-wave 154a in a frequency range from 50 kHz to 1 MHz, e.g., without the use of an amplifier to change the voltage and/or current.

Referring again to FIG. 5B, the processor 170 executes a subroutine (i.e., particular set of processor executable instructions) in a repeated manner so as to generate the modified square-wave 154a. Specifically, for each period 158, the subroutine enables the first circuit 172 and the second circuit 174 to supply and hold the ground signal on the power lines 196 and 199 for a first predetermined period of time 200a, followed by enabling the first circuit 172 to supply (1) a positive voltage with the intermediate amplitude 160a on the power line 196 for a second predetermined period of time 202a, (2) a positive voltage with the amplitude 160 on the power line 196 for a third predetermined period of time 202b, and (3) a positive voltage with the intermediate amplitude 160a on the power line 196 for a fourth predetermined period of time 202c, and the second circuit 174 to supply and hold the ground on the power line 199 for the second, third, and fourth periods of time 202a-c. Then, the subroutine enables the first circuit 172 and the second circuit 174 to supply and hold the ground signals on the power lines 196 and 199 for a sixth predetermined period of time 204a. For simplicity, the modified square-wage 154a for the first half of the period 158 has been described. As detailed above, this subroutine is continued for the remaining time of the period 158 by switching the first circuit 172 and the second circuit 174 and applying a negative voltage in place of the positive voltages in the subroutine. This full subroutine is then repeated to continuously supply the modified square-wave 154a to the first port 180a and the second port 180b, and thus the transducer arrays 70. In some embodiments, the processor executable instructions do not change the duty cycle of the first output signal 182 and the second output signal 184. In these embodiments, the processor 170 does not receive any temperature related feedback from the transducer arrays 70a and 70b, and the processor executable instructions do not include any instructions to modify the signals provided to the first circuit 172 or second circuit 174 based upon temperature related feed back.

### Non-Limiting Illustrative Embodiments of the Inventive Concepts

Illustrative Embodiment 1. A system for generating TTFields, comprising:
a first port operable to connect to a first transducer array;
a second port operable to connect to a second transducer array; and
   an electric field generator having a first circuit generating a first output signal having a positive voltage and a ground voltage; a second circuit generating a second output signal having a negative voltage and a ground voltage; and a processor executing processor executable instructions to alternatingly enable the first output signal, and the second output signal to the first port and the second port to generate an alternating current square wave in a frequency range from 50 kHz to 1 MHz.

Illustrative Embodiment 2. The system of Illustrative Embodiment 1, wherein the first output signal is a direct current square wave having a duty cycle between 15% to 40%, the alternating current square wave having a period, and a positive voltage, a ground voltage and a negative voltage occurring within the period, the positive voltage, the ground voltage and the negative voltage each being held for a predetermined and non-transitory period of time.

Illustrative Embodiment 3. The system of Illustrative Embodiments 1 or 2, wherein the first output signal is a first direct current waveform having a first portion having a first voltage and a second portion having a second voltage lower than the first voltage, the second output signal being a second direct current waveform having a third portion having a third voltage, and a fourth portion having a fourth voltage higher than the third voltage.

Illustrative Embodiment 4. The system of Illustrative Embodiment 3, wherein the first direct current waveform and the second direct current waveform are out of phase such that the first and fourth portions overlap, and the second and third portions overlap.

Illustrative Embodiment 5. The system of Illustrative Embodiment 1, wherein the second output signal is a direct current square wave having a duty cycle between 60% to 85%, the alternating current square wave having a period, and a positive voltage, a ground voltage and a negative voltage occurring within the period, the positive voltage, the ground voltage and the negative voltage of the alternating current square wave each being held for a predetermined and non-transitory period of time.

Illustrative Embodiment 6. The system of any one of Illustrative Embodiments 1, 2, or 5, wherein the electric field generator is devoid of any oscillator circuitry providing a reference signal to the first circuit and the second circuit.

Illustrative Embodiment 7. The system of any one of Illustrative Embodiments 1, 2, or 5, wherein the electric field generator is devoid of any temperature measurement and/or temperature compensation circuitry.

Illustrative Embodiment 8. The system of any one of Illustrative Embodiments 1, 2, or 5, further comprising a battery coupled to the electric field generator, the battery comprising a battery voltage, and wherein the positive voltage of the first output signal is within a range of 5% from the battery voltage.

Illustrative Embodiment 9. The system of Illustrative Embodiment 1, further comprising a third port operable to pass a series of signals indicative of temperature readings to the processor, the processor executable instructions have a temperature compensation subroutine that when executed by the processor, cause the processor to alternatingly enable the first output signal, and the second output signal to the first port and the second port to generate the alternating current square wave in a frequency range from 50 kHz to 1 MHz having at least one non-voltage parameter based upon the temperature reading.

Illustrative Embodiment 10. The system of Illustrative Embodiment 9, wherein the alternating current square wave has a duty cycle, and wherein the non-voltage parameter is the duty cycle, wherein the temperature compensation subroutine, when executed by the processor, causes the processor to vary the duty cycle based upon at least one temperature reading.

Illustrative Embodiment 11. The system of Illustrative Embodiment 10, wherein the temperature compensation subroutine causes the processor to reduce the duty cycle when the at least one temperature reading exceeds a predetermined temperature.

Illustrative Embodiment 12. A method for generating TTFields, the method comprising: alternately enabling a first output signal having a positive voltage from a first circuit and a second output signal having a negative voltage from a second circuit so as to provide an alternating current square wave in a frequency range of 50 kHz and 1 MHz at a first port and a second port of an electric field generator; and supplying the alternating current square wave to transducer arrays mounted on a portion of a patient's body adjacent to a tumor to generate the TTFields.

Illustrative Embodiment 13. The method of Illustrative Embodiment 12, further comprising the step of combining the first output signal and the second output signal so as to provide the alternating current square wave.

Illustrative Embodiment 14. The method of Illustrative Embodiment 13, wherein the first output signal is a first direct current waveform having a first portion having a first voltage and a second portion having a second voltage lower than the first voltage, the second output signal being a second direct current waveform having a third portion having a third voltage, and a fourth portion having a fourth voltage higher than the third voltage, the first direct current waveform and the second direct current waveform being out of phase such that the first and fourth portions overlap, and the second and third portions overlap.

Illustrative Embodiment 15. The method of Illustrative Embodiment 12, further comprising the step of passing a series of signals indicative of temperature readings to a processor of the electric field generator, and wherein the step of alternately enabling is defined further as alternately enabling the first output signal having the positive voltage from the first circuit and the second output signal having the negative voltage from the second circuit so as to provide the alternating current square wave in the frequency range of 50 kHz and 1 MHz at the first port and the second port having at least one non-voltage parameter based upon at least one temperature reading.

Illustrative Embodiment 16. The method of Illustrative Embodiment 15, wherein the alternating current square wave has a duty cycle, and the non-voltage parameter is the duty cycle, and wherein the step of alternately enabling is defined further as alternately enabling the first output signal having the positive voltage from the first circuit and the second output signal having the negative voltage from the second circuit so as to provide the alternating current square wave in the frequency range of 50 kHz and 1 MHz at the first port and the second port having a duty cycle based upon at least one temperature reading.

Illustrative Embodiment 17. The method of Illustrative Embodiment 16, further comprising the step of reducing the duty cycle when the at least one temperature reading exceeds a predetermined temperature.

Illustrative Embodiment 18. A system for generating TTFields, comprising:
a first transducer array having a first lead;
a second transducer array having a second lead;
an electronic field generator comprising:
a first port operable to receive the first lead of the first transducer array;
a second port operable to receive the second lead of the second transducer array;
a first circuit generating a first output signal having a positive voltage;
a second circuit generating a second output signal having a negative voltage; and
a processor executing processor executable instructions to alternatingly enable the first output signal, and the second output signal to the first port and the second port to generate an alternating current square wave in a frequency range from 50 kHz to 1 MHz between the first transducer array and the second transducer array when the first transducer array and the second transducer array are affixed to a patient's body.

Illustrative Embodiment 19. The system of Illustrative Embodiment 18, wherein the first output signal is a direct current square wave having a duty cycle between 15% to 40%.

Illustrative Embodiment 20. The system of Illustrative Embodiments 18 or 19, wherein the first output signal is a first direct current waveform having a first portion having a first voltage and a second portion have a second voltage lower than the first voltage, the second output signal being a second direct current waveform having a third portion having a third voltage, and a fourth portion having a fourth voltage higher than the third voltage.

Illustrative Embodiment 21. The system of Illustrative Embodiment 20, wherein the first direct current waveform and the second direct current waveform are out of phase such that the first and fourth portions overlap, and the second and third portions overlap.

Illustrative Embodiment 22. The system of Illustrative Embodiment 18, wherein the second output signal is a direct current square wave having a duty cycle between 60% to 85%.

Illustrative Embodiment 23. The system of any one of Illustrative Embodiments 18, 19, or 22, wherein the electric field generator is devoid of any oscillator circuitry providing a reference signal to the first circuit and the second circuit.

Illustrative Embodiment 24. The system of any one of Illustrative Embodiments 18, 19, or 22, wherein the electric field generator is devoid of any temperature measurement and/or temperature compensation circuitry.

Illustrative Embodiment 25. The system of any one of Illustrative Embodiments 18, 19, or 22, further comprising a battery coupled to the electric field generator, the battery comprising a battery voltage, and wherein the positive voltage of the first output signal is within a range of 5% from the battery voltage.

Illustrative Embodiment 26. The system of Illustrative Embodiment 25, wherein the battery has a voltage in a range from 20V to 40V.

Illustrative Embodiment 27. The system of Illustrative Embodiment 18, wherein the electric field generator further comprises a third port operable to pass a series of signals indicative of temperature readings to the processor, and wherein the processor executable instructions have a temperature compensation subroutine that when executed by the processor, cause the processor to alternatingly enable the first output signal, and the second output signal to the first port and the second port to generate the alternating current square wave in a frequency range from 50 kHz to 1 MHz having at least one non-voltage parameter based upon the temperature reading.

Illustrative Embodiment 28. The system of Illustrative Embodiment 27, wherein the alternating current square wave has a duty cycle, and wherein the non-voltage parameter is the duty cycle, wherein the temperature compensation subroutine, when executed by the processor, causes the processor to vary the duty cycle based upon at least one temperature reading.

Illustrative Embodiment 29. The electric field generator of Illustrative Embodiment 28, wherein the temperature compensation subroutine causes the processor to reduce the duty cycle when the at least one temperature reading exceeds a predetermined temperature.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

From the above description and examples, it is clear that the inventive concepts disclosed and claimed herein are well adapted to attain the advantages mentioned herein. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

What is claimed is:

1. A system for generating TTFields, comprising:
a first port operable to connect to a first transducer array;
a second port operable to connect to a second transducer array; and
an electric field generator having a first circuit generating a first output signal having a positive voltage and a ground voltage; a second circuit generating a second output signal having a negative voltage and a ground voltage; and a processor executing processor executable instructions to alternatingly enable the first output signal, and the second output signal to the first port and the second port to generate an alternating current square wave in a frequency range from 50 kHz to 1 MHz.

2. The system of claim 1, wherein the first output signal is a direct current square wave having a duty cycle between 15% to 40%, the alternating current square wave having a period, and a positive voltage, a ground voltage and a negative voltage occurring within the period, the positive voltage, the ground voltage and the negative voltage each being held for a predetermined and non-transitory period of time.

3. The system of claim 2, wherein the first output signal is a first direct current waveform having a first portion having a first voltage and a second portion having a second voltage lower than the first voltage, the second output signal being a second direct current waveform having a third portion having a third voltage, and a fourth portion having a fourth voltage higher than the third voltage.

4. The system of claim 3, wherein the first direct current waveform and the second direct current waveform are out of phase such that the first and fourth portions overlap, and the second and third portions overlap.

5. The system of claim 1, wherein the second output signal is a direct current square wave having a duty cycle between 60% to 85%, the alternating current square wave having a period, and a positive voltage, a ground voltage and a negative voltage occurring within the period, the positive voltage, the ground voltage and the negative voltage of the alternating current square wave each being held for a predetermined and non-transitory period of time.

6. The system of claim 1, wherein the electric field generator is devoid of any oscillator circuitry providing a reference signal to the first circuit and the second circuit.

7. The system of claim 1, wherein the electric field generator is devoid of any temperature measurement and/or temperature compensation circuitry.

8. The system of claim 1, further comprising a battery coupled to the electric field generator, the battery comprising a battery voltage, and wherein the positive voltage of the first output signal is within a range of 5% from the battery voltage.

9. A method for generating TTFields, the method comprising:
alternately enabling a first output signal having a positive voltage from a first circuit and a second output signal having a negative voltage from a second circuit so as to provide an alternating current square wave in a frequency range of 50 kHz and 1 MHz at a first port and a second port of an electric field generator; and
supplying the alternating current square wave to transducer arrays mounted on a portion of a patient's body adjacent to a tumor to generate the TTFields.

10. The method of claim 9, further comprising the step of combining the first output signal and the second output signal so as to provide the alternating current square wave.

11. The method of claim 10, wherein the first output signal is a first direct current waveform having a first portion having a first voltage and a second portion having a second voltage lower than the first voltage, the second output signal being a second direct current waveform having a third portion having a third voltage, and a fourth portion having a fourth voltage higher than the third voltage, the first direct current waveform and the second direct current waveform being out of phase such that the first and fourth portions overlap, and the second and third portions overlap.

12. A system for generating TTFields, comprising:
a first transducer array having a first lead;
a second transducer array having a second lead;
a first port operable to receive the first lead of the first transducer array;
a second port operable to receive the second lead of the second transducer array; and
an electric field generator having a first circuit generating a first output signal having a positive voltage; a second circuit generating a second output signal having a negative voltage; and a processor executing processor executable instructions to alternatingly enable the first output signal, and the second output signal to the first port and the second port to generate an alternating current square wave in a frequency range from 50 kHz to 1 MHz between the first transducer array and the second transducer array when the first transducer array and the second transducer array are affixed to a patient's body.

13. The system of claim 12, wherein the first output signal is a direct current square wave having a duty cycle between 15% to 40%.

14. The system of claim 13, wherein the first output signal is a first direct current waveform having a first portion having a first voltage and a second portion have a second voltage lower than the first voltage, the second output signal being a second direct current waveform having a third portion having a third voltage, and a fourth portion having a fourth voltage higher than the third voltage.

15. The system of claim 14, wherein the first direct current waveform and the second direct current waveform are out of phase such that the first and fourth portions overlap, and the second and third portions overlap.

16. The system of claim 12, wherein the second output signal is a direct current square wave having a duty cycle between 60% to 85%.

17. The system of claim 12, wherein the electric field generator is devoid of any oscillator circuitry providing a reference signal to the first circuit and the second circuit.

18. The system of claim 12, wherein the electric field generator is devoid of any temperature measurement and/or temperature compensation circuitry.

19. The system of claim 12, further comprising a battery coupled to the electric field generator, the battery comprising a battery voltage, and wherein the positive voltage of the first output signal is within a range of 5% from the battery voltage.

20. The system of claim 19, wherein the battery has a voltage in a range from 20V to 40V.
